(19) United States
(12) Patent Application Publication
(10) Pub. No.: US 2024/0207560 A1
(43) Pub. Date: Jun. 27, 2024

(54) VENTILATORS ADAPTORS

(71) Applicant: THE ROYAL INSTITUTION FOR THE ADVANCEMENT OF LEARNING/MCGILL UNIVERSITY, Montreal (CA)

(72) Inventors: Kosar KHWAJA, Pierrefonds (CA); Robert E. KEARNEY, Montreal (CA); Ross WAGNER, Saint-Leonard (CA); Orhun KOSE, Montreal (CA)

(21) Appl. No.: 17/914,113

(22) PCT Filed: Mar. 23, 2021
(86) PCT No.: PCT/CA2021/050382

§ 371 Date: Sep. 23, 2022

Related U.S. Application Data
(60) Provisional application No. 62/993,599, filed on Mar. 23, 2020.

(51) Int. Cl. A61M 16/08; A61M 16/00; A61M 16/12

(52) U.S. Cl. CPC A61M 16/0816; A61M 16/0003; A61M 16/022; A61M 16/12; A61M 2205/3368; A61M 2205/505

(57) ABSTRACT

A ventilator adaptor for converting a ventilator having an inspiratory port and an expiratory port from single patient use to multiple patient use is disclosed. The ventilator adaptor includes an inspiratory splitter, and expiratory splitter and a controller. The inspiratory splitter is attachable to the inspiratory port of the ventilator, and at least two inspiratory branches which are each connectable to a different patient. The expiratory splitter is attachable to the expiratory port of the ventilator, and at least two expiratory branches which are each connectable to the respective different patient. The controller separately controls at least one inspiratory parameter of an inspired fluid in each of the at least two inspiratory branches and/or at least one expiratory parameter of an expired fluid in each of the at least two expiratory branches. A ventilator system having the ventilator adaptor, and methods for ventilating at least two patients are also disclosed.

### FIELD OF THE DISCLOSURE

The present disclosure relates to ventilator adaptors for enabling the connection of multiple patients to a single ventilator.

### BACKGROUND OF THE DISCLOSURE

Ventilators provide mechanical breathing assistance to patients by supplying an input fluid into a patient's lungs and removing an output fluid from the patient's lungs. The inlet fluid can be air with enhanced oxygen levels and may also include other gaseous components such as drugs.

Conventional ventilators comprise an oxygen regulator for modulating a concentration of oxygen in the input gas (expressed, for example as FiO2), a humidification system for modulating a water vapour level in the input gas; and a flow device for controlling the flow rate, pressure and/or volume of the input gas and/or the output gas. Ventilators may also include nebulizer components for delivering drugs to the patient. A patient interface, which can be a mask or an endotracheal tube, fluidly connects the ventilator to an airway of the patient via an inspiratory tube, for conducting the input gas, or an expiratory tube for conducting the output gas (FIGS. 1 and 2). A processor of the ventilator can control one or more parameters of the ventilator such as the input or output gas flow rates, pressure, volume, input gas humidity, input gas oxygen concentration etc. These parameters can be controlled via the ventilator and can be tailored to each individual patient and their given condition and requirements. The ventilator may include, or be connected to, sensors, such as for monitoring various physiological parameters of the patient for example expiratory oxygen and carbon dioxide levels, as well as heart rate (FIG. 2).

In certain situations, health systems, hospitals and clinics may face an increased need for ventilators, such as during pandemics or other increases in respiratory conditions by the human population.

### SUMMARY OF THE DISCLOSURE

It is an object of the present technology to ameliorate at least some of the inconveniences present in the prior art.

Developers of the present technology have appreciated that simply splitting inspiratory and expiratory tubes of a ventilator system into multiple respective inspiratory and expiratory tubes for multiple patients do not provide the ability to adapt the various parameters that can be controlled by the ventilator (e.g. pressure, volume, flow, oxygen concentration and humidity levels) per patient. This can be dangerous and sometimes life threatening for those patients.

Broadly, aspects of the present technology provide adaptors for ventilators which can provide a separate control of inhaled and exhaled fluids to different patients, thereby permitting multiple patients to be ventilated simultaneously from a single ventilator.

According to one aspect of the present technology, there is provided a ventilator adaptor for converting a ventilator having an inspiratory port and an expiratory port to multiple patient use. The ventilator adaptor includes an inspiratory splitter attachable to the inspiratory port of the ventilator, and an expiratory splitter attachable to the expiratory port of the ventilator. The ventilator adaptor also includes at least two breathing circuits. Each breathing circuit is connectable to the inspiratory splitter and the expiratory splitter to define a given breathing circuit for a given patient. The ventilator adaptor also includes a controller for separately controlling at least one parameter of a fluid in the at least two breathing circuits. Each breathing circuit comprises an inspiratory portion and an expiratory portion and the controller is configured to control the at least one parameter in one or both of the inspiratory portion and the expiratory portion. The at least one parameter is one or more of fluid pressure, fluid flow rate, fluid temperature, and fluid humidity. The ventilator adaptor may include one or more sensors for detecting one or more of fluid pressure, fluid flow rate, fluid temperature, and fluid humidity in the fluid in the at least two breathing circuits. The controller and/or the one or more sensors may be operatively coupleable to a processor of a computer system.

According to another aspect of the present technology, there is provided a ventilator adaptor for converting a ventilator having an inspiratory port and an expiratory port from single patient use to multiple patient use. The ventilator adaptor comprises an inspiratory splitter, an expiratory splitter, and a controller. The inspiratory splitter is attachable to the inspiratory port of the ventilator, and at least two inspiratory branches which are each connectable to a different patient. The expiratory splitter is attachable to the expiratory port of the ventilator, and at least two expiratory branches which are each connectable to the respective different patient. The controller separately controls one or both of at least one inspiratory parameter of an inspired fluid in each of the at least two inspiratory branches and at least one expiratory parameter of an expired fluid in each of the at least two expiratory branches.

In some embodiments, the controller is configured to separately control the at least one inspiratory parameter.

In some embodiments, the at least one inspiratory parameter is fluid pressure, and the controller comprises at least one pressure regulator configured to separately modulate the fluid pressure of the inspired fluid in each of the at least two inspiratory branches.

In some embodiments, the controller is configured to separately modulate the fluid pressure of the inspired fluid in each of the at least two inspiratory branches to a maximum fluid pressure. In some embodiments, the maximum fluid pressure is 50 cmH₂0.

In some embodiments, the controller is configured to maintain a baseline fluid pressure of the inspired fluid in each of the at least two inspiratory branches between 5 to 15 cmH₂0. In some embodiments, the baseline fluid pressure is maintained in 5 cm H₂0 increments.

In some embodiments, the controller is communicatively connectable to a processor of a computer system.

In some embodiments, the ventilator adaptor further includes a display for displaying at least one of the at least one inspiratory parameter, and/or the at least one expiratory parameter and the at least one expiratory parameter. In some embodiments, the display is communicatively connectable to one or both of the controller and the processor. The display may comprise a touchscreen.

In some embodiments, the at least one inspiratory parameter is fluid pressure of the inspired fluid (e.g. PIP), and the at least one expiratory parameter is fluid pressure of the expired fluid (e.g. (Positive Expiratory End Pressure (PEEP)), and the controller is configured to separately control the fluid pressure of the inspired fluid and the expired fluid in each of the at least one inspiratory branches and each of the at least one expiratory branches.

In some embodiments, the fluid is a gas. In some embodiments, the fluid is a mixture of medical air and oxygen.

In some embodiments, the inspiratory tube is arranged to be fluidly connectable to an inspiratory port of the ventilator, and the single expiratory tube is arranged to be fluidly connectable to an expiratory port of the ventilator.

In some embodiments, a first one of the at least two inspiratory branches and a first one of the at least two expiratory branches are fluidly connectable to a first one of the at least two patients, and a second one of the at least two inspiratory branches and a second one of the at least two expiratory branches are fluidly connectable to a second one of the at least two patients.

In some embodiments, the ventilator adaptor further includes one or both of: at least one inspiratory sensor for monitoring the at least one inspiratory parameter in the at least two inspiratory branches, and at least one expiratory sensor for monitoring the at least one expiratory parameter in the at least two expiratory branches.

In some embodiments, the at least one inspiratory sensor and/or the at least one expiratory sensor is connected to one or both of the controller and the processor.

In some embodiments, the at least one inspiratory parameter is a pressure, and the at least one inspiratory sensor comprises an inspiratory pressure sensor for monitoring the pressure of the inspiratory fluid in the at least two inspiratory branches.

In some embodiments, the at least one expiratory parameter is a pressure, and the at least one expiratory sensor comprises an expiratory pressure sensor for monitoring the pressure of the expiratory fluid in the at least two expiratory branches. The controller and/or the expiratory pressure sensor may be configured to maintain a Positive Expiratory End Pressure (PEEP) of the expired fluid at a pressure of at least 5 cm H₂O.

In some embodiments, the at least one inspiratory parameter is a flow rate. The at least one inspiratory sensor may include an inspiratory flow sensor for monitoring the flow rate of the inspiratory fluid in the at least two inspiratory branches.

In some embodiments, the at least one expiratory parameter is a flow rate. The at least one expiratory sensor may include an expiratory flow sensor for monitoring the flow rate of the expiratory fluid in the at least two expiratory branches.

In some embodiments, the at least one inspiratory parameter is a gas volume. The at least one inspiratory sensor may include an inspiratory volume sensor for monitoring the gas volume of the inspiratory fluid. In certain embodiments, the gas volume may be derived from other direct measurements.

In some embodiments, the at least one expiratory parameter is a gas volume. The at least one expiratory sensor may include an expiratory volume sensor for monitoring the gas volume of the expiratory fluid. In certain embodiments, the gas volume may be derived from other direct measurements.

In some embodiments, the at least one inspiratory parameter is a temperature of the fluid. The at least one inspiratory sensor may include an inspiratory temperature sensor for monitoring the temperature of the inspiratory fluid.

In some embodiments, the at least one expiratory parameter is a temperature of the fluid. The at least one expiratory sensor may include an expiratory temperature sensor for monitoring the temperature of the expiratory fluid.

In some embodiments, the at least one inspiratory parameter is a humidity. The at least one inspiratory sensor may include an inspiratory humidity sensor for monitoring the humidity of the inspiratory fluid.

In some embodiments, the at least one expiratory parameter is a humidity. The at least one expiratory sensor may include an expiratory humidity sensor for monitoring the humidity of the expiratory fluid.

In some embodiments, the ventilator adaptor further includes an inspiratory one-way valve in at least one of the at least two inspiratory branches.

In some embodiments, the ventilator adaptor further includes an expiratory one-way valve in at least one of the at least two expiratory branches.

In some embodiments, the ventilator adaptor further includes an inspiratory viral filter connected to at least one of the at least two inspiratory branches.

In some embodiments, the ventilator adaptor further includes an expiratory viral filter connected to at least one of the at least two expiratory branches.

In some embodiments, the ventilator adaptor further includes a positive end expiratory pressure valve connected to at least one of the at least two expiratory branches.

In some embodiments, the ventilator adaptor further includes a housing. The housing includes therein at least a portion of the at least two inspiratory branches, at least a portion of the at least two expiratory branches, and at least a portion of the controller.

According to another aspect of the present technology, there is provided a ventilator system including a ventilator having an inspiratory port and an expiratory port, and the ventilator adaptor according to the above aspect or according to the above aspect and one or more of the above embodiments.

In some embodiments of the ventilator system, a first one of the at least two inspiratory branches and a first one of the at least two expiratory branches are fluidly connectable to a first one of the two patients to define a first breathing circuit, and a second one of the at least two inspiratory branches and a second one of the at least two expiratory branches are fluidly connectable to a second one of the two patients to define a second breathing circuit, the first and second breathing circuits being separate from one another.

According to another aspect of the present technology, there is provided a method for ventilating at least two patients, the at least two patients being connected to a ventilator having a inspiratory port and a expiratory port by a ventilator adaptor, the ventilator adaptor configured to adapt the single input port and the expiratory port to provide a first breathing circuit to a first patient of the at least two patients and a second breathing circuit to a second patient of the at least two patients, the first and second breathing circuits being separate from each other. The method may be at least partially executed by a processor of a computer system. The method includes detecting a value of an inspiratory parameter of an inspired fluid and/or or an expiratory parameter of an expired fluid, in each of the first breathing circuit and the second breathing circuit. After detecting the value of the inspiratory parameter and/or the expiratory parameter, the method includes causing a modulation of the value of the inspiratory parameter and/or the expiratory parameter according to a desired value. The modulation may be performed manually by an operator, or automatically by the processor according to predetermined rules.

In some embodiments, after causing the modulation of the value of the inspiratory parameter and/or the expiratory parameter according to the desired value, the method further includes detecting the value of the inspiratory parameter and/or the expiratory parameter.

In some embodiments, after detecting the value of the inspiratory parameter and/or the expiratory parameter, the method further includes causing display of the value of the inspiratory parameter and/or the expiratory parameter on a display.

According to another aspect of the present technology, there is provided a method for ventilating at least two patients, the at least two patients being connected to a ventilator having a inspiratory port and a expiratory port by a ventilator adaptor, the ventilator adaptor configured to adapt the single input port and the expiratory port to provide a first breathing circuit to a first patient of the at least two patients and a second breathing circuit to a second patient of the at least two patients, the first and second breathing circuits being separate from each other. The method may be at least partially executed by a processor of a computer system. The method includes detecting a value of an inspiratory parameter of an inspired fluid and/or an expiratory parameter of an expired fluid in each of the first breathing circuit and the second breathing circuit. After detecting the value of the inspiratory parameter and/or the expiratory parameter, the method includes causing a display of the detected value.

From another aspect, there is provided a ventilator adaptor which is fluidly coupleable to a ventilator, and which is arranged to: split an inspiratory circuit into at least two inspiratory circuits and provide a means for controlling a parameter of a gas in each of the at least two inspiratory circuits; and split an expiratory circuit into at least two expiratory circuits and provide a means for controlling a parameter of a gas in each of the at least two expiratory circuits.

From yet another aspect, there is provided a ventilator adaptor which is fluidly coupleable to a ventilator, and which is arranged to: split an inspiratory tube into at least two inspiratory branches and provide a means for controlling a parameter of a gas in each of the at least two inspiratory branches; and split an expiratory tube into at least two expiratory branches and provide a means for controlling a parameter of a gas in each of the at least two expiratory branches.

In certain embodiments, the ventilator adaptor is arranged to be fluidly couplable to an inspiratory port and an expiratory port of the ventilator. In certain embodiments, the adaptor further comprises an inspiratory connector for connecting the adaptor to the inspiratory port; and an expiratory connector for connecting the adaptor to the expiratory port.

In certain embodiments, the controller is arranged to control a parameter of a gas flow through one of the at least two inspiratory or expiratory branches or circuits.

In certain embodiments, the parameter is a gas pressure in one of the at least inspiratory branches or circuits, and the controller is a pressure regulator.

In certain embodiments, the controller further comprises a one-way valve.

In certain embodiments, the controller is a flow sensor to measure a flow rate of a gas in one of the at least two inspiratory branches or inspiratory circuits.

In certain embodiments, the controller is a volume sensor to measure a volume of a

gas in one of the at least two expiratory branches or expiratory circuits.

In certain embodiments, the adaptor comprises a housing including therein: the controller; at least a portion of the at least two inspiratory branches or at least a portion of the at least two inspiratory circuits; and at least a portion of the at least two expiratory branches or at least a portion of the at least two expiratory circuits.

From another aspect, there is provided a ventilator adaptor which is fluidly coupleable to a ventilator, and which is arranged to: split an inspiratory circuit into at least two inspiratory circuits and provide a means for controlling a parameter of a gas in each of the at least two inspiratory circuits; and split an expiratory circuit into at least two expiratory circuits and provide a means for controlling a parameter of a gas in each of the at least two expiratory circuits.

From another broad aspect, there is provided a ventilator adaptor which is fluidly coupleable to a ventilator, and which is arranged to: split an inspiratory tube into at least two inspiratory branches and provide a means for controlling a parameter of a gas in each of the at least two inspiratory branches; and split an expiratory tube into at least two expiratory branches and provide a means for controlling a parameter of a gas in each of the at least two expiratory branches.

In certain embodiments, the ventilator adaptor is arranged to be fluidly couplable to an inspiratory port and an expiratory port of the ventilator. In certain embodiments, the adaptor further comprises an inspiratory connector for connecting the adaptor to the inspiratory port; and an expiratory connector for connecting the adaptor to the expiratory port.

In certain embodiments, the controller is arranged to control a parameter of a gas flow through one of the at least two inspiratory or expiratory branches or circuits.

In certain embodiments, the parameter is a gas pressure in one of the at least inspiratory branches or circuits, and the controller is a pressure regulator.

In certain embodiments, the controller further comprises a one-way valve.

In certain embodiments, the controller is a flow sensor to measure a flow rate of a gas in one of the at least two inspiratory branches or inspiratory circuits.

In certain embodiments, the controller is a volume sensor to measure a volume of a gas in one of the at least two expiratory branches or expiratory circuits.

In certain embodiments, the adaptor comprises a housing including therein: the controller; at least a portion of the at least two inspiratory branches or at least a portion of the at least two inspiratory circuits: at least a portion of the at least two expiratory branches or at least a portion of the at least two expiratory circuits.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

### BRIEF DESCRIPTION OF DRAWINGS

Further aspects and advantages of the present technology will become better understood with reference to the description in association with the following in which:

FIG. 1 is a block diagram of a ventilator system of the prior art comprising a ventilator connected to a single patient.

FIG. 2 is a schematic illustration of the ventilator of FIG. 1.

FIG. 3 is a block diagram of a ventilator system comprising a ventilator adaptor connected to a ventilator and two patients, according to certain embodiments of the present technology.

FIG. 4 is a schematic illustration of the ventilator system of FIG. 3 showing the ventilator adaptor in more detail, according to certain embodiments of the present technology.

FIG. 5 is a schematic illustration of the ventilator adaptor of FIG. 3 connected to the ventilator of FIG. 3 and two patients, according to certain embodiments of the present technology.

FIG. 6 is a schematic illustration of the ventilator adaptor of FIG. 3 connected to the ventilator of FIG. 3 and two patients, according to certain embodiments of the present technology.

FIG. 7 is a schematic illustration of the ventilator adaptor of FIG. 3 connected to the ventilator of FIG. 3 and two patients, according to certain embodiments of the present technology.

FIG. 8 is a schematic illustration of a portion of the ventilator adaptor of FIG. 3 connected to the ventilator of FIG. 1 and two patients, according to certain embodiments of the present technology.

FIG. 9 is a portion of a display of the ventilator adaptor of FIG. 3 according to certain embodiments of the present technology.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

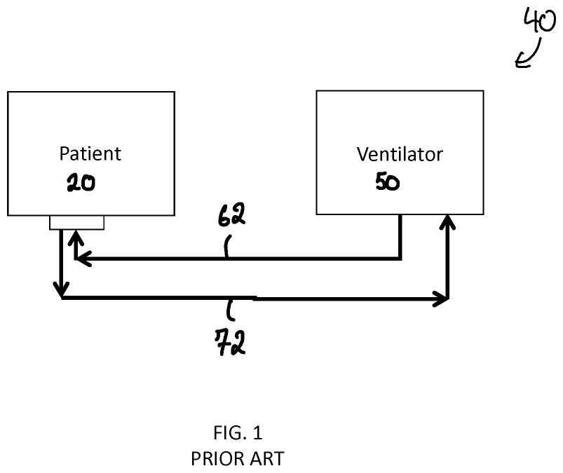

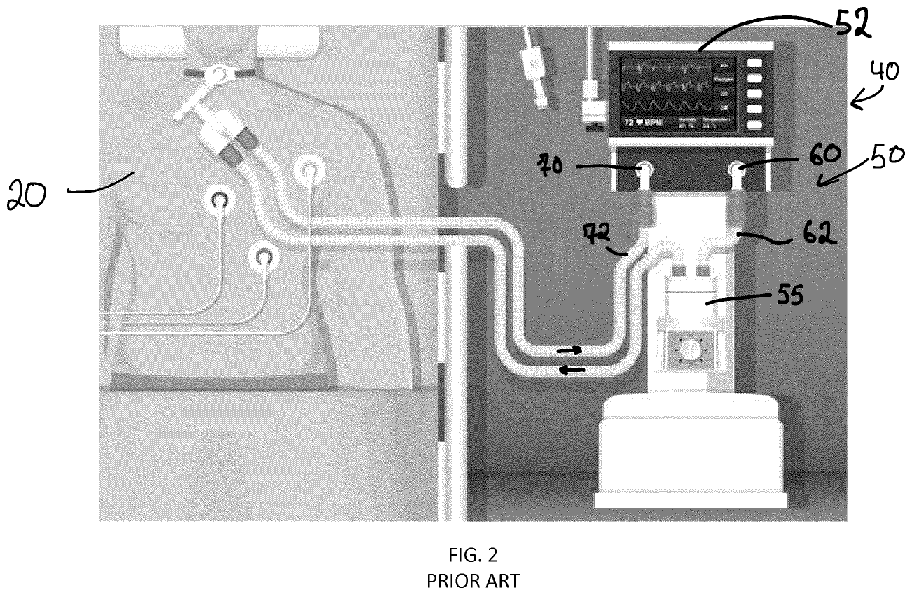

A ventilator system 40 comprising a ventilator 50 connectable to a patient, as known in the art, is shown in FIGS. 1 and 2. Broadly, the ventilator 50 is configured to be fluidly connected to one single patient, patient 20, to help the said patient 20 breathe. The ventilator system 40 has a ventilator display 52 that displays information associated with the ventilator 50. The ventilator has an inspiratory port 60 that is fluidly connected to an inspiratory tube 62 through which fluid flows towards the patient 20, and an expiratory port 70 that is fluidly connected to an expiratory tube 72 through which fluid flows away from the patient 20. The ventilator 50 of the prior art may also have a humidifier 55 (shown in FIG. 2) that is fluidly connected to the inspiratory tube 62, and that can modulate the humidity and/or temperature of the fluid in the inspiratory tube 62. The ventilator 50 may also have a fluid inlet (not shown) through which fluid can be supplied to the ventilator 50, for further supply to the patient 20. The fluid can be air which may be itself be enhanced, such as with oxygen or medication.

In use, the inspiratory and expiratory tubes 62, 72 are fluidly connected to the patient 20, thereby forming a respiratory circuit with an inspiratory portion and an expiratory portion. In the inspiratory portion, an inspired fluid, such as the fluid supplied to the ventilator 50 flows from the ventilator 50 to the patient 20, and in the expiratory portion, an expiratory fluid flows from the patient 20 to the ventilator 50. The inspired and/or expired fluid is typically controlled so as to modulate one or more of a composition, a pressure, a flow rate, a humidity, and/or a temperature of the inspired fluid. Each of these fluid parameters are specific for a given patient at a given moment in time, and may need to be adjusted over time. Therefore, prior art ventilator systems 40 are limited to connecting to a single patient at a time, in order to be able to provide a set of inspired and/or expired fluid properties which are tailored for the given patient.

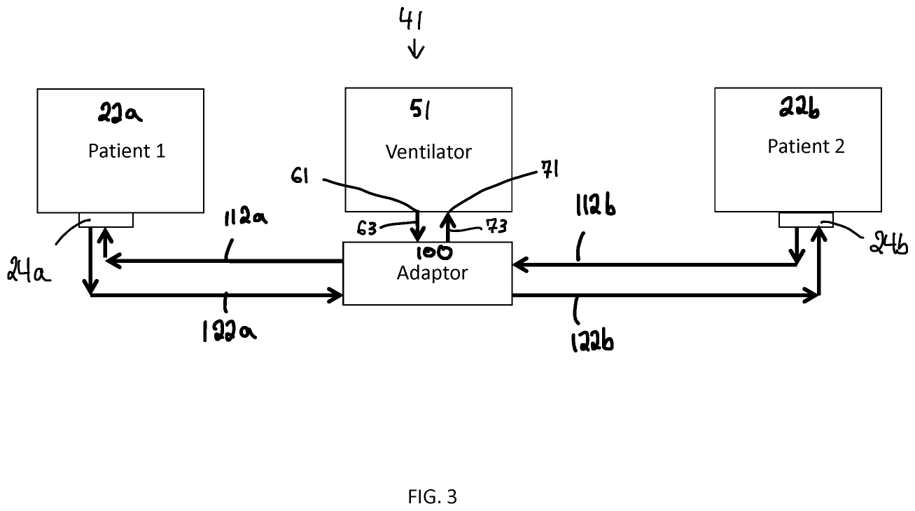

According to certain aspects and embodiments of the present technology, and with reference to FIGS. 3 to 8, there are provided ventilator systems 41 that permit the ventilation of more than one patient at a time and in a manner in which each patient can be provided with a tailored set of inspired fluid properties and/or a tailored set of expired fluid parameters. In other words, inspired and/or expired fluid properties can be independently controlled for multiple patients simultaneously, according to certain embodiments. Each patient can be provided with the fluid at a suitable pressure.

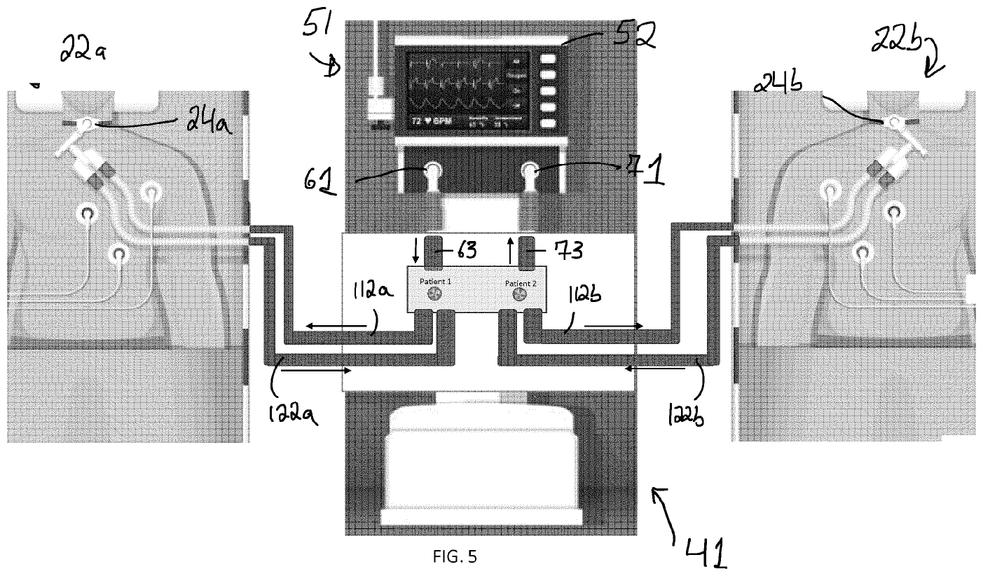

Referring initially to FIGS. 3 and 5, according to certain embodiments, the ventilator system 41 comprises a ventilator adaptor 100 (also referred to herein as “adaptor”). The ventilator adaptor 100 is configured to fluidly connect a ventilator 51 having an inspiratory port 61 and expiratory port 71 to two or more patients via an inspiratory tube 63 and an expiratory tube 73, respectively. In such certain embodiments, the adaptor 100 is configured to be fluidly connected to the ventilator 50 of the prior art by the inspiratory port 60 and the expiratory port 70.

Advantageously, the adaptor 100 can be retrofit to any type of ventilator having an inspiratory port and expiratory port to increase the number of patients that can be ventilated using a single ventilator. In embodiments in which the ventilator 51 is a single patient use ventilator, the adaptor 100 converts the ventilator 51 from single patient use to multi patient use. In the embodiments illustrated and described herein, the adaptor 100 is configured to assist two patients, patient 22a and patient 22b. However, in other embodiments, the adaptor 100 is configured to fluidly connect three or more patients to the ventilator 51. The adaptor 100 is configured to assist mechanical ventilation in a manner that allows independent control of at least one gas parameter to and/or from the ventilator 51. For example, a composition of the fluid may be modulated by means of a carbon dioxide or oxygen line fluidly connected to the ventilator 51.

The adaptor 100 has an inspiratory splitter 110 that is attachable to the inspiratory port 61 via a fluid conduit such as the inspiratory tube 63. Extending from the inspiratory splitter 110, and fluidly connected thereto, are a plurality of inspiratory fluid conduits, inspiratory branches 112a, 112b, each configured to be fluidly connectable to a given patient. The adaptor 100 also has an expiratory splitter 120 that is attachable to the expiratory port 71 of the respirator 51 via a fluid conduit such as the expiratory tube 73. Extending from the expiratory splitter 120, and fluidly connected thereto, are a plurality of expiratory fluid conduits, expiratory branches 122a, 122b, each configured to be fluidly connectable to a given patient. As such, the inspiratory tube 63 is mechanically converted to the two inspiratory branches 112a, 112b by the inspiratory splitter 110, and similarly, the single expiratory tube 73 is mechanically converted to the two expiratory branches 122a, 122b by the expiratory splitter 110. It will be appreciated that in some embodiments, the inspiratory splitter 110 is attachable to the inspiratory port 61 directly, without requiring the inspiratory tube 63. In certain embodiments, the expiratory splitter 120 is attachable to the expiratory port 71, without requiring the expiratory tube 73.

As it will be appreciated, the adaptor 100 is thus configured to define separate breathing circuits for the different patients by means of the inspiratory splitter 110 and the expiratory splitter 120. More specifically, in use, when the inspiratory branch 112a and the expiratory branch 122a are connected to the patient 22a, there is defined a first breathing circuit. Similarly, when the inspiratory branch 112b and the expiratory branch 122b are connected to the patient 22b, in use, there is defined a second breathing circuit. The first and/or the second breathing circuits may include fluid input via the ventilator 51. By breathing circuit is meant a looped fluid pathway between the patient and the adaptor 100. It will be appreciated that there may be provided one or more ventilation circuits which further encompass fluid pathways between the ventilator 51 and the adaptor 100.

The adaptor 100 is further provided with a controller 130 for independently monitoring and/or controlling certain parameters of the fluid in the different breathing circuits, such as the first and second breathing circuit and more specifically in one or more of the inspiratory branches 112a, 112b, and the expiratory branches 122a, 122b. For example, the controller 130 is configured to monitor and/or control at least one inspiratory parameter of the fluid in the inspiratory branches 112a, 112b in certain embodiments. The controller 130 monitors and/or controls at least one expiratory parameter of the fluid in the expiratory branches 122a, 122b in certain embodiments. It is contemplated that in some embodiments, the controller 130 is configured to control the at least one inspiratory parameter only i.e. without monitoring and/or controlling the at least one expiratory parameter, or vice versa. In this respect, the controller 130 may be operatively connectable to a processor of a computer system (not shown) for its operation thereof. The computer system may be associated with the adaptor 100, the ventilator 51 or with any other equipment. The controller 130 may be configured to send data regarding the at least one inspiratory parameter and/or the at least one expiratory parameter to a memory associated with the computer system. The controller 130 may also be configured to receive physiological data associated with the patients, and in this respect may be communicatively coupled to one or more device for collecting the physiological data. It is contemplated that in some embodiments, the controller 130 could have different sub-components for controlling separately the inspired fluid and the expired fluid. The controller 130 may be configured to control other fluid parameters such as one or more of: flow rate, pressure, volume, temperature and/or humidity. In other embodiments, the at least one expiratory parameter and/or the at least one inspiratory parameter may be controlled manually, such as by an operator. In certain embodiments, the controller 130 is configured to generate a signal on detection of a predetermined inspiratory parameter value and/or an expiratory parameter value. The signal may be a visual, sound or haptic notification.

The adaptor 100 is further provided with a housing 102 in which at least a portion of the inspiratory branches 112a, 112b, at least a portion of the expiratory branches 122a, 122b and the controller 130 are housed.

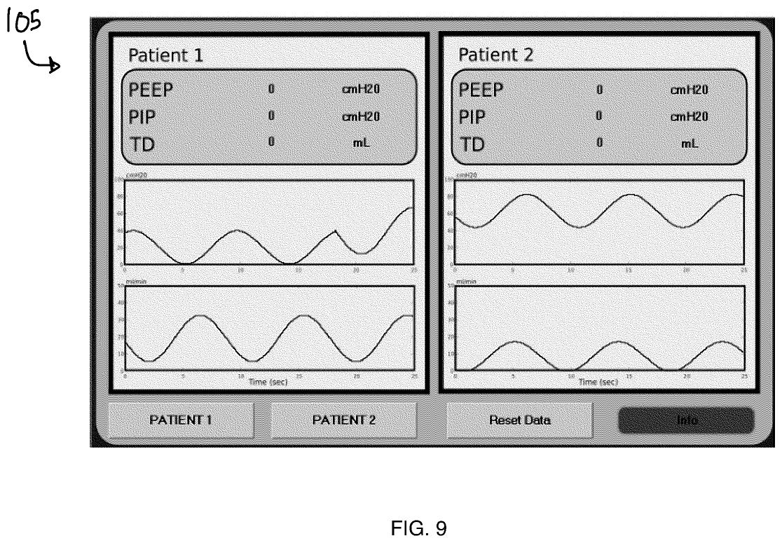

In certain embodiments, the adaptor 100 is further provided with a display 105 (partially shown in FIG. 9) that is communicatively connectable to the controller 130. The display 105 has a touch screen, although the touch screen could be omitted in other embodiments. It is contemplated that in some embodiments, the display 105 could be communicatively connectable to the processor of the computer system, if present. As will be described in greater detail below, the display 105 is configured to display inspiratory and/or expiratory parameters of the inspired and/or expired fluids as well as the values of the said inspiratory and/or expiratory parameters. The display 105 may permit two-way communication between an operator 105 and the controller 130.

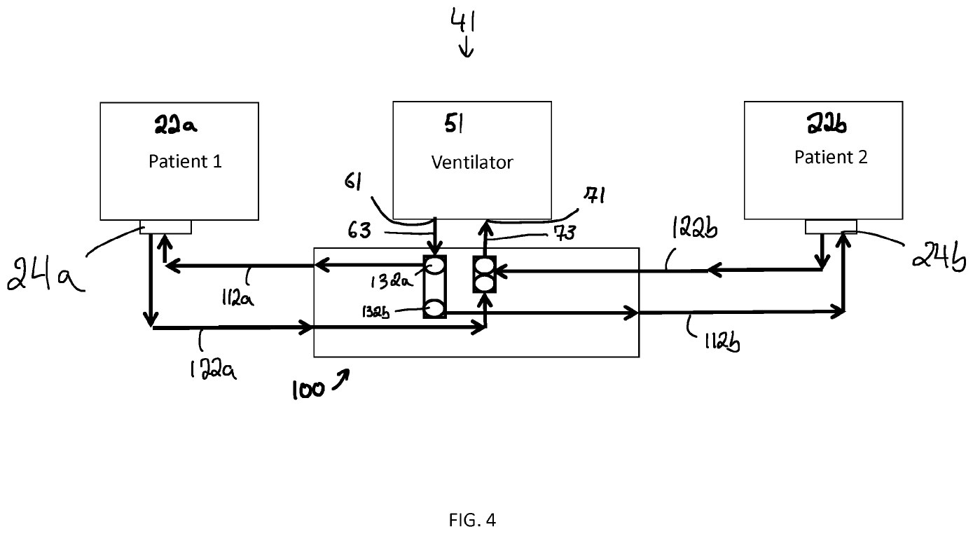

As best seen in FIGS. 4, 6 and 7, in certain embodiments, the controller 130 includes pressure regulators 132a, 132b which are respectively connected to the inspiratory branches 112a, 112b. The pressure regulator 132a controls fluid pressure of the inspired fluid in the inspiratory branch 112a, and the pressure regulator 132b controls fluid pressure of the inspired fluid in the inspiratory branch 112b. In other embodiments (not shown), a single pressure regulator separately controls fluid pressure in the inspiratory branches 112a, 112b. In this respect, at least one of the inspiratory parameters is fluid pressure. The pressure regulators 132a, 132b may include pressure sensors. In certain embodiments, the controller 130 or the ventilation system may also include other types of sensors such as flow sensors 200a, 200b

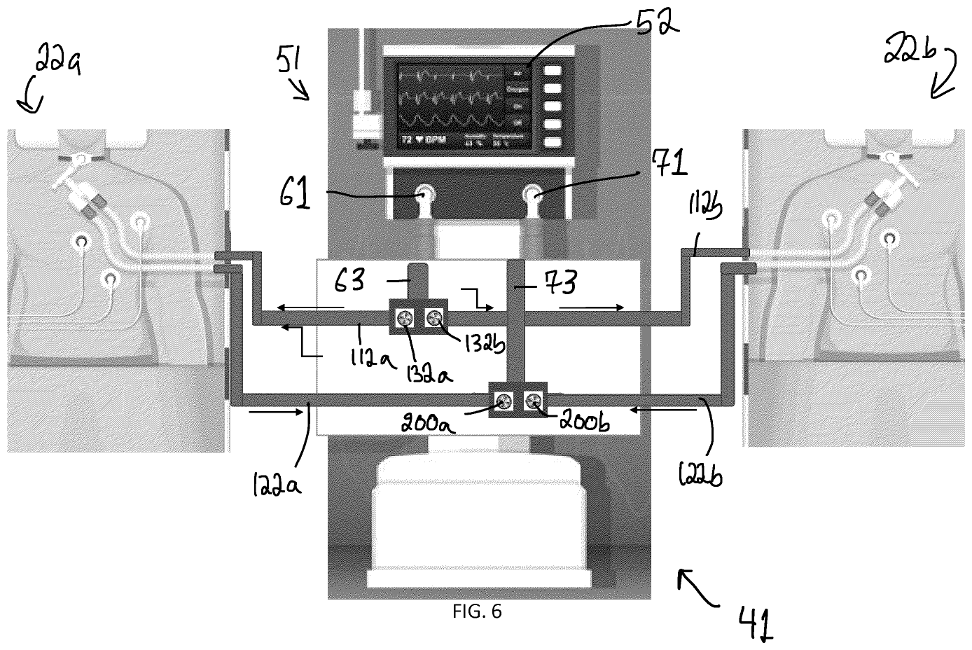

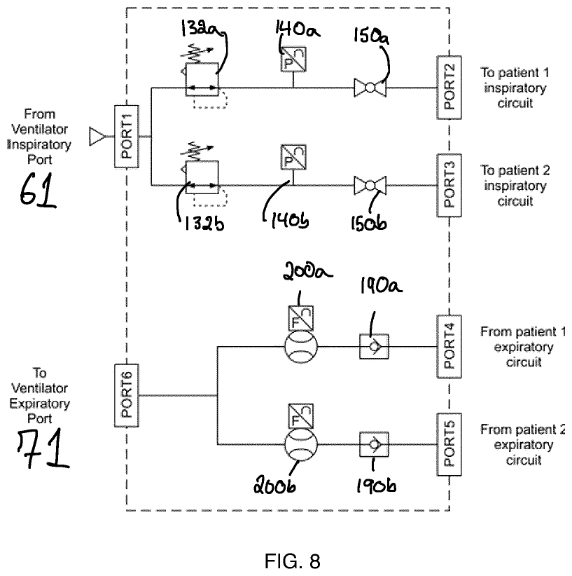

(FIGS. 6 and 8).

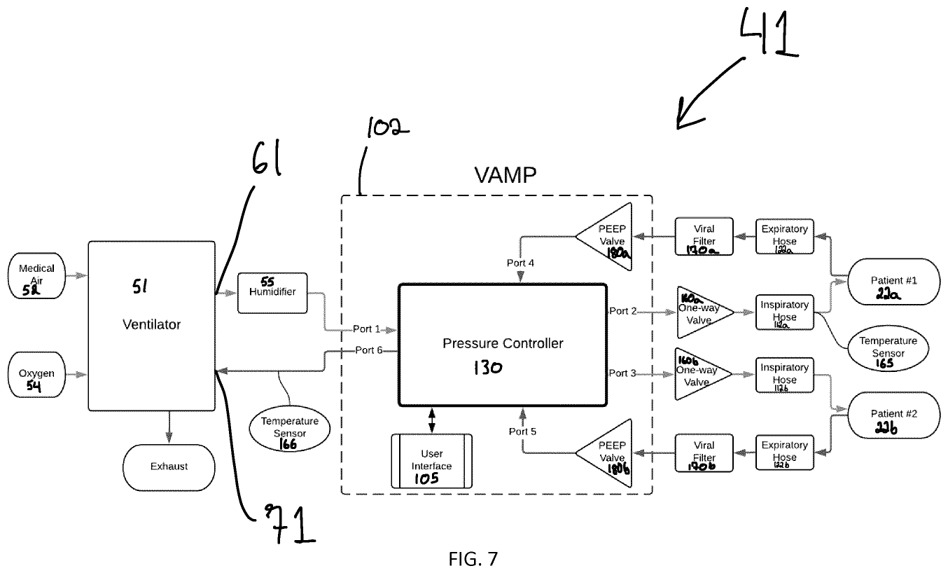

Referring particularly to FIGS. 7 and 8, an inspiratory portion of the respiratory circuits for patients 22a, 22b will now be described in greater detail, flowing from the inspiratory tube 63 to the patients 22a, 22b. It will be appreciated that any of the components described below may be included within the housing 102 of the adaptor 100 or external thereto. Those components external to the housing 102 may be considered as part of the ventilator system 41 or as part of the adaptor 100.

Pressure sensors 140a, 140b are provided that are respectively connected to the inspiratory branches 112a, 112b, and positioned downstream from the pressure regulators 132a, 132b. The pressure sensors 140a, 140b are also electronically connected to the controller 130, and by extension to the processor of the computer system. The pressure sensors 140a, 140b can monitor the fluid pressure of the inspired fluid in the inspiratory branches 112a, 112b. Thus, the pressure sensors 140a, 140b can be considered as an inspiratory sensor. The fluid pressures monitored by the pressure sensors 140a, 140b may be displayed on the display 105. It is contemplated that in other embodiments, other sensors may be provided such as flow sensors, volume sensors, temperature sensors and/or humidity sensors.

Ball valves 150a, 150b are provided that are respectively connected to the inspiratory branches 112a, 112b, and positioned downstream from the sensors 140a, 140b. The ball valves 150a, 150b permit one of the inspiratory branches 112a, 112b to be disconnected without affecting fluid flow through the other one of the inspiratory branches 112a, 112b.

One-way valves 160a, 160b are provided that are respectively connected to the inspiratory branches 112a, 112b, and positioned downstream from the ball valves 150a, 150b. The one-way valves 160a, 160b ensure that fluid flows in a given direction in the inspiratory branches 112a, 112b, the given direction being from the ventilator 51 to the patients 22a, 22b.

A temperature sensor 165 is connected to the inspiratory branch 112a, and positioned downstream from the one-way valve 160a. It is contemplated that in other embodiments, the temperature sensor 165 could be connected to the inspiratory branch 112b. In yet other embodiments, there could be more than one temperature sensor 165, that could be connected to both of the inspiratory branches 112a, 112b. The temperature sensor 165 monitors the temperature of the inspired fluid in the inspiratory branch 112a. Thus, the temperature sensor 165 can be considered as an inspiratory sensor and at least one of the inspiratory parameters is fluid temperature. The monitored temperature can be used to control the humidifier 55. In some embodiments, the temperature monitored by the temperature sensor 165 may be displayed on the display 105.

The inspiratory branch 112a is connected to the patient 22a by a patient interface 24a, and the inspiratory branch 112b is connected to the patient 22b by a patient interface 24b, which in the embodiment of FIG. 5 is illustrated as invasive. In other embodiments, the patient interface may comprise non-invasive ventilation masks.

In some embodiments (not shown), inspiratory viral filters may be provided connected to the inspiratory portions of the respiratory circuits.

Still referring particularly to FIGS. 7 and 8, an expiratory portion of the adaptor 100 will now be described, flowing in a direction from the patients 22a, 22b to the expiratory port 71.

Like the inspiratory branches 112a, 112b, the expiratory branch 122a is connected to the patient 22a by the patient interface 24a, and the expiratory branch 122b is connected to the patient 222 by the patient interface 24b.

The adaptor 100 has expiratory viral filters 170a, 170b that are respectively connected to the expiratory branches 122a, 122b, and that are positioned downstream from the patients 22a, 22b. The expiratory viral filters 170a, 170b may have any suitable configuration and pore size for filtering virus's and other contaminants and can aid in minimizing possibilities of cross-infections.

Positive end expiratory pressure [PEEP] valves 180a, 180b, which may be adjustable, are provided and are respectively connected to the expiratory branches 122a, 122b, and that are positioned downstream from the expiratory viral filters 170a, 170b. The PEEP valves 180a, 180b allow the expired fluid to return to the ventilator 51 only when the fluid pressures of the expired fluid in the expiratory branches 122a, 122b are above a baseline fluid pressure set by the PEEP valves 180a, 180b. Furthermore, the PEEP valves 180a, 180b act as one-way valves.

Check valves 190a, 190b are provided which are respectively connected to the expiratory branches 122a, 122b, and are positioned downstream from the PEEP valves 180a, 180b. With the check valves 190a, 190b, one of the expiratory branches 122a, 122b can be disconnected without affecting fluid flow through the other one of the expiratory branches 122a, 122b.

Also provided are flow sensors 200a, 200b that are respectively connected to the expiratory branches 122a, 122b, and that are positioned downstream from the check valves 190a, 190b. The flow sensors 200a, 200b are also electronically connected to the controller 130. As mentioned above, it is contemplated that in some embodiments, the flow sensors 200a, 200b could be connected to another separate controller. It is contemplated that in other embodiments, the flow sensors 200a, 200b could be electronically connected to a processor of a computer system. The flow sensors 200a, 200b monitor the flow rate of the expired fluid in the expiratory branches 122a, 122b. Thus, the flow sensors 200a, 200b can be considered as expiratory sensors and at least one of the expiratory parameters is flow rate. The flow rates monitored by the flow sensors 200a, 200b are displayed on the display 105. It is contemplated that in other embodiments, the sensors 200a, 200b could be other sensors such as pressure sensors, volume sensors, temperature sensors and/or humidity sensors.

Finally, the expiratory splitter 120 connects the expiratory branches 122a, 122b to the single expiratory tube 73 and to the expiratory port 71. The expiratory branches 122a, 122b, may have any suitable configuration for conveying fluid therethrough.

It is contemplated that in some embodiments, the adaptor 100 could include the pressure regulators 132a, 132b, the pressure sensors 140a, 140b, the ball valves 150a, 150b, the check valves 190a, 190b and the flow sensors 200a, 200b as part of the controller 130. It is contemplated that in some embodiments, however, some of the features are separate from the controller 130.

The housing, in addition to the portion of the inspiratory branches 112a, 112b, the portion of the expiratory branches 122a, 122b and the controller 130, as mentioned above, also includes therein the pressure sensors 140a, 140b, the ball valves 150a, 150b, the PEEP valves 180a, 180b, and the check valves 190a, 190b and the flow sensors 200a, 200b, in certain embodiments. The display 105 may be external to the housing but connected thereto. The housing 102 is useful, for making the adaptor 100 portable, transportable, and protecting the components housed therein. The housing 102 may also permit ease of cleaning and/or sterilization. It is contemplated that in other embodiments, the housing 102 could include more or less features therein. For instance, the expiratory viral filters 170a, 170b could also be included in the housing 102.

A temperature sensor 166, which is connected to the humidifier 55, is connected to the single expiratory tube 73, and is positioned upstream from the ventilator 51. The temperature sensor 166 monitors the temperature of the expired fluid in the single expiratory tube 73 which temperature may be communicated to the humidifier 55 by a processor. In some embodiments, the temperature monitored by the temperature sensor 166 is displayed on the display 105. In this respect, at least one of the expiratory parameter is temperature.

It is understood that the arrangement of some of the features mentioned above (e.g. PEEP valve being downstream from the expiratory viral filter) can be rearranged without departing from the scope of the present technology.

An operation of the ventilator system 41 will now be described. As described above, the ventilator 51 is fed air 52 and oxygen 54 (schematically shown in FIG. 7), which is then mixed at a precise ratio to form the inspired fluid. The inspired fluid is a gas. It is contemplated that in other embodiments, the inspired fluid could be mixture of other gases. The inspired fluid flows from the inspiratory port 61 to the inspiratory tube 63.

The inspired fluid then flows through the humidifier 55, which has a humidity sensor therein that monitors the humidity of the inspired fluid. The humidifier 55 controls the humidity and the temperature of the inspired fluid according to a predetermined value, which may be different from patient to patient, and can be set and controlled. The inspired fluid is then split into the inspiratory branches 112a, 112b by the inspiratory splitter 110.

The pressure regulator 132a modulates the fluid pressure of the inspired fluid in the inspiratory branch 112a depending on the ventilation support required by the patient 22a. Similarly, the pressure regulator 132b modulates the fluid pressure of the inspired fluid in the inspiratory branch 112b depending on the ventilation support required by the patient 22b. The fluid pressure in each of the inspiratory branches 112a, 112b can be manually adjusted by an operator using the display 105. In some embodiments, the fluid pressures can be adjusted automatically by the controller 130. The pressure regulators 132a, 132b can modulate the fluid pressure within a range of 20 to 50 cmH₂O. In the present embodiment, the maximum fluid pressure that can be provided by the pressure regulators 132a, 132b is 50 cmH₂O. It is contemplated that in some embodiments, the pressure regulators 132a, 132b could be configured to have different ranges and/or different maximum values.

The inspired fluid in each of the inspiratory branches 112a, 112b is then monitored by the pressure sensors 140a, 140b. The processor and/or controller 130 may obtain the pressure value(s) of the inspired fluid and can subsequently cause these values to be displayed on the display 105. The fluid pressure in each of the inspiratory branches 112a, 112b may be displayed separately on the display 105.

The inspired fluid then flows past the ball valves 150a, 150b and past the one-way valves 160a, 160b which prevent back flow. The temperature of the inspired fluid of the inspiratory branch 112a is then monitored by the temperature sensor 165. The temperature monitored by the temperature sensor 165 may be communicated to the humidifier 55 which can adjust the humidity upstream.

Thus, the inspired fluid eventually reaches the patients 22a, 22b. The pressure differences between the inspiratory branches 112a, 112b and the lungs of the patients 22a, 22b first force the inspired fluid into the lungs of the patients 22a, 22b. Then, when the pressure difference drops, the expired fluid is expired by the lungs and enters the expiratory branches 122a, 122b.

The expired fluid flows past the expiratory viral filters 170a, 170b until the PEEP valves 180a, 180b are reached. As mentioned above, the PEEP valves 180a, 180b are adjustable and set the baseline fluid pressure. In the present embodiment, the baseline fluid pressure is between 5 to 15 cmH₂O, and is maintained in 5 cmH₂O increments. It is contemplated that in other embodiments, the baseline fluid pressure properties could be different.

Then, the expired fluid flows past the check valves 190a, 190b and reaches the flow sensors 200a, 200b in each of the expiratory branches 122a, 122b. The flow sensors 200a, 200b monitor the flow rates in the expiratory branches 122a, 122b, and communicate the flow rates to the controller 130, which are subsequently communicated to the display 105, such that the flow rates of both of the expiratory branches 122a, 122b are displayed on the display 105.

Then, the expired fluid from the expiratory branches 122a, 122b converges to the single expiratory tube 74 by the expiratory splitter 120, reaches the temperature sensor 166, before finally reaching the ventilator 51, where the expired air is exhausted.

A method for ventilating the two patients 22a, 22b that are connected to the ventilator 51 by the adaptor 100, and executed by a processor of a computer system will now be described. As mentioned above, the adaptor 100 is configured to adapt the inspiratory port 61 and the expiratory port 71 to provide a first breathing circuit to the patient 22a, and a second breathing circuit to the patient 22b. It is contemplated that in some embodiments, the method could be executed by the controller 130.

The method includes detecting a value of an inspiratory parameter of the inspired fluid in the inspiratory branches 112a, 112b upstream from the pressure regulators 132a, 132b. In the present embodiment, the inspiratory parameter is fluid pressure. It is contemplated that in other embodiments, a value of another inspiratory parameter such as one or more of: flow rate, volume, temperature and/or humidity could be detected. In some embodiments, the detected value from this step is displayed on the display 105.

Then, the method includes modulating the value of the detected inspiratory parameter to a desired value. In the present embodiment, the desired value is set by the processor. It is contemplated that in other embodiments, the desired value could be set by the controller 130. In yet other embodiments, the desired value could be set by an operator using the display 105. The inspiratory parameter being fluid pressure, the fluid pressure of the inspired fluid in the inspiratory branches 112a, 112b is modulated by the controller 130.

Then, the method includes causing a display of the detected value of the previous step on the display 105.

Then, the method includes detecting a value of an inspiratory parameter of the inspired fluid in the inspiratory branches 112a, 112b downstream from the pressure regulators 132a, 132b. The inspiratory parameter is fluid pressure. It is contemplated that in other embodiments, a value of another inspiratory parameter such as one or more of: flow rate, volume, temperature and/or humidity could be detected.

Then, the method includes causing a display of the detected value of the previous step on the display 105.

In certain non-limiting embodiments of the present technology, the computer system is thus configured to receive and/or send data and/or instructions between the controller, the operator and the ventilator 51. According to some non-limiting embodiments of the present technology, the computer system may receive the data via local input/output interface (such as USB, as an example, not separately depicted). In other non-limiting embodiments of the present technology, the computer system may be configured to receive the data over a communication network, to which the computer system is communicatively coupled.

In some non-limiting embodiments of the present technology, the communication network is the Internet and/or an Intranet. Multiple embodiments of the communication network may be envisioned and will become apparent to the person skilled in the art of the present technology. Further, how a communication link between the computer system and the communication network is implemented will depend on how the computer system is implemented, and may include, but is not limited to, a wire-based communication link and a wireless communication link (such as a Wi-Fi communication network link, a 3G/4G communication network link, and the like).

Further, a computing environment suitable for use with some implementations of the present technology may comprise various hardware components including the processor (single or multi-core), a solid-state drive, a random-access memory and an input/output interface. Communication between the various components of the computing environment may be enabled by one or more internal and/or external buses (e.g. a PCI bus, universal serial bus, IEEE 1394 “Firewire” bus, SCSI bus, Serial-ATA bus, ARINC bus, etc.), to which the various hardware components are electronically coupled. The input/output interface allows enabling networking capabilities such as wire or wireless access. As an example, the input/output interface comprises a networking interface such as, but not limited to, a network port, a network socket, a network interface controller and the like. Multiple examples of how the networking interface may be implemented will become apparent to the person skilled in the art of the present technology. For example, but without being limiting, the input/output interface 580 may implement specific physical layer and data link layer standard such as Ethernet™, Fibre Channel, Wi-Fi™ or Token Ring™. The specific physical layer and the data link layer may provide a base for a full network protocol stack, allowing communication among small groups of computers on the same local area network (LAN) and large-scale network communications through routable protocols, such as IP.

According to implementations of the present technology, the solid-state drive stores program instructions suitable for being loaded into the random-access memory and executed by the processor, according to certain aspects and embodiments of the present technology. For example, the program instructions may be part of a library or an application.

In some non-limiting embodiments of the present technology, the computing environment is implemented in a generic computer system, which is a conventional computer (i.e. an “off the shelf” generic computer system). The generic computer system may be a desktop computer/personal computer, but may also be any other type of electronic device such as, but not limited to, a laptop, a mobile device, a smart phone, a tablet device, or a server.

As persons skilled in the art of the present technology may appreciate, multiple variations as to how the computing environment can be implemented may be envisioned without departing from the scope of the present technology.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the technology is not limited to the particular embodiments described and illustrated herein but includes all modifications and variations falling within the scope of the technology as defined in the appended claims.

1. A ventilator adaptor for converting a ventilator having an inspiratory port and an expiratory port from single patient use to multiple patient use, the ventilator adaptor comprising:
an inspiratory splitter attachable to the inspiratory port of the ventilator, and at least two inspiratory branches which are each connectable to a different patient,
an expiratory splitter attachable to the expiratory port of the ventilator, and at least two expiratory branches which are each connectable to the respective different patient; and
a controller for separately controlling one or both of at least one inspiratory parameter of an inspired fluid in each of the at least two inspiratory branches and at least one expiratory parameter of an expired fluid in each of the at least two expiratory branches.

2. The ventilator adaptor of claim 1, wherein the controller is configured to separately control the at least one inspiratory parameter.

3. The ventilator adaptor of claim 1 or claim 2, wherein the at least one inspiratory parameter is fluid pressure, the controller comprising at least one pressure regulator configured to separately modulate the fluid pressure of the inspired fluid in each of the at least two inspiratory branches.

4. The ventilator adaptor of claim 3, wherein the controller is configured to separately modulate the fluid pressure of the inspired fluid in each of the at least two inspiratory branches to a maximum fluid pressure.

5. The ventilator adaptor of claim 4, wherein the maximum fluid pressure is 50 cmH₂0.

6. The ventilator adaptor of any one of claims 1 to 5, wherein the controller is configured to maintain a baseline fluid pressure of the inspired fluid in each of the at least two inspiratory branches between 5 to 15 cmH₂0.

7. The ventilator adaptor of claim 6, wherein the baseline fluid pressure is maintained in 5 cm H₂0 increments.

8. The ventilator adaptor of any one of claims 1 to 7, wherein the controller is communicatively connectable to a processor of a computer system.

9. The ventilator adaptor of any one of claims 1 to 8, further comprising a display for displaying at least one of the at least one inspiratory parameter and the at least one expiratory parameter.

10. The ventilator adaptor of claim 9 when dependent on claim 8, wherein the display is communicatively connectable to the controller.

11. The ventilator adaptor of claim 9 or claim 10, wherein the display comprises a touchscreen.

12. The ventilator adaptor of any one of claims 1 to 11, wherein the at least one inspiratory parameter is fluid pressure of the inspired fluid, and the at least one expiratory parameter is fluid pressure of the expired fluid, the controller being configured to separately control the fluid pressure of the inspired fluid and the expired fluid in each of the at least one inspiratory branches and each of the at least one expiratory branches.

13. The ventilator adaptor of any one of claims 1 to 12, wherein the fluid is a gas.

14. The ventilator adapter of any one of claims 1 to 13, wherein the fluid is a mixture of medical air and oxygen.

15. The ventilator adaptor of any one of claims 1 to 14, further comprising an inspiratory tube for fluidly connecting the inspiratory splitter to the inspiratory port of the ventilator, and an expiratory tube for fluidly connecting the expiratory splitter to the expiratory port of the ventilator.

16. The ventilator adapter of any one of claims 1 to 15, wherein:
a first one of the at least two inspiratory branches and a first one of the at least two expiratory branches are fluidly connectable to a first one of the at least two patients; and
a second one of the at least two inspiratory branches and a second one of the at least two expiratory branches are fluidly connectable to a second one of the at least two patients.

17. The ventilator adaptor of any one of claims 1 to 16, further comprising one or both of:
at least one inspiratory sensor for monitoring the at least one inspiratory parameter in the at least two inspiratory branches, and at least one expiratory sensor for monitoring the at least one expiratory parameter in the at least two expiratory branches.

18. The ventilator adaptor of claim 17, wherein the at least one inspiratory sensor and/or the at least one expiratory sensor is connected to the controller.

19. The ventilator adaptor of claim 18 when dependent on claim 8, wherein the at least one inspiratory sensor and/or the at least one expiratory sensor is connected to the processor.

20. The ventilator adaptor of any one of claims 17 to 19, wherein the at least one inspiratory parameter is a pressure, the at least one inspiratory sensor comprising an inspiratory pressure sensor for monitoring the pressure of the inspiratory fluid in the at least two inspiratory branches.

21. The ventilator adaptor of any one of claims 17 to 20, wherein the at least one expiratory parameter is a pressure, the at least one expiratory sensor comprising an expiratory pressure sensor for monitoring the pressure of the expiratory fluid in the at least two expiratory branches.

22. The ventilator adaptor of claims 17 to 21, wherein the at least one inspiratory parameter is a gas volume, further comprising an inspiratory volume sensor for monitoring the gas volume of the inspiratory fluid.

23. The ventilator adaptor of any one of claims 17 to 22, wherein the at least one expiratory parameter is a gas volume, further comprising an expiratory volume sensor for monitoring the gas volume of the expiratory fluid.

24. The ventilator adaptor of any one of claims 17 to 23, wherein the at least one inspiratory parameter is a temperature of the fluid, and further comprising an inspiratory temperature sensor for monitoring the temperature of the inspiratory fluid.

25. The ventilator adaptor of any one of claims 17 to 24, wherein the at least one expiratory parameter is a temperature of the fluid, and further comprising an expiratory temperature sensor for monitoring the temperature of the expiratory fluid.

26. The ventilator adaptor of any one of claims 17 to 25, wherein the at least one inspiratory parameter is a humidity, and further comprising an inspiratory humidity sensor for monitoring the humidity of the inspiratory fluid.

27. The ventilator adaptor of any one of claims 17 to 26, wherein the at least one expiratory parameter is a humidity, and further comprising an expiratory humidity sensor for monitoring the humidity of the expiratory fluid.

28. The ventilator adaptor of any one of claims 1 to 27, further comprising an inspiratory one-way valve in at least one of the at least two inspiratory branches.

29. The ventilator adaptor of any one of claims 1 to 28, further comprising an expiratory one-way valve in at least one of the at least two expiratory branches.

30. The ventilator adaptor of any one of claims 1 to 29, further comprising an inspiratory viral filter connected to at least one of the at least two inspiratory branches.

31. The ventilator adaptor of any one of claims 1 to 30, further comprising an expiratory viral filter connected to at least one of the at least two expiratory branches.

32. The ventilator adaptor of any one of claims 1 to 31, further including a positive end expiratory pressure valve connected to at least one of the at least two expiratory branches.

33. The ventilator adaptor of any one of claims 1 to 32, further comprising a housing, the housing including therein:
at least a portion of the at least two inspiratory branches;
at least a portion of the at least two expiratory branches; and
the controller.

34. A ventilator adaptor for converting a ventilator having an inspiratory port and an expiratory port from single patient use to multiple patient use, the ventilator adaptor comprising:
an inspiratory splitter attachable to the inspiratory port of the ventilator, and an expiratory splitter attachable to the expiratory port of the ventilator;
at least two breathing circuits, each breathing circuit connectable to the inspiratory splitter and the expiratory splitter to define a given breathing circuit for a given patient; and
a controller for separately controlling at least one parameter of a fluid in the at least two breathing circuits.

35. A ventilator system comprising:
a ventilator having an inspiratory port and an expiratory port, and
the ventilator adaptor of any one of claims 1 to 33.

36. The ventilator system of claim 35, wherein:
a first one of the at least two inspiratory branches and a first one of the at least two expiratory branches are fluidly connectable to a first one of the two patients to define a first breathing circuit; and
a second one of the at least two inspiratory branches and a second one of the at least two expiratory branches are fluidly connectable to a second one of the two patients to define a second breathing circuit, the first and second breathing circuits being separate from one another.

37. A method for ventilating at least two patients, the at least two patients being connected to a ventilator having an inspiratory port and a expiratory port by a ventilator adaptor, the ventilator adaptor configured to adapt the single input port and the expiratory port to provide a first breathing circuit to a first patient of the at least two patients and a second breathing circuit to a second patient of the at least two patients, the first and second breathing circuits being separate from each other, the method being executed by a processor of a computer system, the method comprising:
detecting a value of one or both of an inspiratory parameter of an inspired fluid and an expiratory parameter of an expired fluid in each of the first breathing circuit and the second breathing circuit;
causing a modulation of the value of the inspiratory parameter according to a desired value.

38. A method for ventilating at least two patients, the at least two patients being connected to a ventilator having an inspiratory port and a expiratory port by a ventilator adaptor, the ventilator adaptor configured to adapt the single input port and the expiratory port to provide a first breathing circuit to a first patient of the at least two patients and a second breathing circuit to a second patient of the at least two patients, the first and second breathing circuits being separate from each other, the method being executed by a processor of a computer system, the method comprising:
detecting a value of one or both of an inspiratory parameter of an inspired fluid and an expiratory parameter of an expired fluid in each of the first breathing circuit and the second breathing circuit;
causing a display of the detected value on a display.
